(19) United States
(12) Patent Application Publication
(10) Pub. No.: US 2014/0088687 A1
(43) Pub. Date: Mar. 27, 2014

(54) VASCULAR ENDOPROSTHESIS

(71) Applicants: George L. De Goicoechea, Pinecrest, FL (US); Juan Carlos Parodi, Buenos Aires (AR); Guro Bjørnstad, Cassis (FR)

(72) Inventors: George L. De Goicoechea, Pinecrest, FL (US); Juan Carlos Parodi, Buenos Aires (AR); Guro Bjørnstad, Cassis (FR)

(73) Assignee: ST. GEORGE MEDICAL INC., Pinecrest, FL (US)

(21) Appl. No.: 14/116,480

(22) PCT Filed: May 10, 2012
(86) PCT No.: PCT/FR2012/051038

§ 371 Date: Nov. 8, 2013

(30) Foreign Application Priority Data
May 10, 2011 (FR) 1154036

(51) Int. Cl. A61F 2/07; A61B 19/00; A61F 2/856

(52) U.S. Cl. CPC A61F 2/07; A61F 2/856; A61B 19/54
USPC 623/1.13; 623/1.35; 623/1.34

(57) ABSTRACT

The invention relates to a vascular endoprosthesis including a device for preventing any endoleak after an angioplasty. The invention more particularly but not restrictively relates to a vascular endoprosthesis for a patient suffering from an abdominal aortic aneurysm.

### CROSS REFERENCE TO RELATED APPLICATIONS

This application is the U.S. National Phase Application of PCT/FR2012/051038, filed May 10, 2012, which claims priority to French Application No. 1154036, filed May 10, 2011, the contents of such applications being incorporated by reference herein.

### FIELD OF THE INVENTION

The invention relates to a vascular endoprosthesis, or more generally to a device intended to be introduced into a blood vessel, the walls of which are aneurysmal, so that blood stops circulating in said vessel proper and can circulate through the endoprosthesis. The invention more particularly but not restrictively relates to a vascular endoprosthesis for a patient suffering from an abdominal aortic aneurysm.

### BACKGROUND OF THE INVENTION

An aneurysm of the abdominal aorta is a serious pathology which generally results in a permanent local dilatation of the infrarenal abdominal aorta. Such dilatation may exceed 50% of the nominal diameter and generally goes together with the loss of parallelism of the bag-shaped (saccular aneurysm) or spindle-shaped (fusiform aneurysm) edges thereof. The progressive growth of such an aneurysm is inevitable and difficult to predict. The application of a femoral introduced vascular endoprosthesis to be fixed to the artery walls upstream and downstream of the aneurysm using stents (metal or polymer structures) is currently one of the main techniques for the treatment of an abdominal aortic aneurysm, a technique which is considered less invasive than a surgical treatment.

Known endoprostheses mainly consist of a generally metallic tubular or bifurcated structure the metal mesh (stent) of which is covered with a thin synthetic membrane. Such endoprosthesis is introduced into the femoral artery in the inguinal fold. It is generally folded in a tube or a sheath—sometimes called an introducer—which moves forward inside the vessel up to the aneurysm, where it is unfolded. The metallic structure, or even additional means for fixing it to the aorta wall normally prevent any migration of the endoprosthesis and prevent blood from circulating in the cavity of the aneurysm or aneurysmal sac. The endoprosthesis can be automatically unfolded when removing the sheath (spring effect of the metal mesh—stent) or using a balloon also introduced into said sheath which is inflated on demand to expand the metal mesh of the endoprosthesis. This technique is particularly efficient, more particular in case of loss of parallelism of the infrarenal aorta edges. Whatever the technique used, the current endoprosthesis cannot however ensure a correct tightness at the anchoring point of the endoprosthesis between the outer wall of the endoprosthesis and the inner wall of the abdominal aorta. The development of the pathology may, in particular, create endoleaks, which result in blood flowing, over time, into the aneurysmal sac, thus causing certain and serious complications.

Besides, the known endoprosthesis give the practitioner only few teachings when they are positioned or during the patient's follow-up as to the positioning thereof within the pathological vessel. This often means an additional difficulty for the practitioner when making the treatment act or taking care of the patient.

In addition, the known endoprosthesis are generally composed of an integral stent (metal mesh) covered with a membrane. This is more particularly the case for endoprosthesis which are applied using a sheath stressing said stent and the removal of which causes the automatic expansion of the stent so that it can take shape within the vessel. This structure involves a significant manufacturing cost and some rigidity which may be prejudicial, depending on the configuration of the aneurysm to be treated.

### SUMMARY OF THE INVENTION

Aspects of the invention addresses all the disadvantages raised by the known solutions. The invention mainly consists in providing expandable means of the fabric or polymer types, positioned as an outer ring on the upper distal portion of the tubular member of the endoprosthesis, so that said means prevents all endoleaks. In the preferred application for the treatment of an abdominal aortic aneurysm, said expandable means adheres to the inner wall of the aorta after the expansion of an upper stent upstream of the aneurysmal sac and downstream of the renal arteries. Thanks to the invention, all operative complications can be eliminated.

Among the numerous advantages brought by the invention, we can mention that the invention makes it possible to:
limit the number of mechanical parts in the simplest endoprosthesis, thus reducing the manufacturing costs and thus increasing the reliability and flexibility of the assembly;
prevent any risk of leaks on and migration of the prosthesis;
help the practitioner—when making the treatment act or taking care of the patient—in particular by using radiopaque means.

For this purpose, a vascular endoprosthesis is provided which includes a substantially tubular main member, the upper distal portion of which cooperates with a stent. In order to prevent all risks of endoleaks, the invention provides that said endoprosthesis includes expandable fabric means covering the lower portion of said stent and cooperating with the upper distal portion of said member.

According to a preferred embodiment, the tubular member may consist of woven polyester.

The stent may further be expandable, not retractable, and consist of a chrome-cobalt- and nitinol-based metal mesh. As an alternative solution, said stent may consist of a metal mesh made of stainless steel.

According to the invention, the expandable means may advantageously consist of a knitted material including a resilient synthetic fibre.

In order to extend the main member of an endoprosthesis according to the invention, if need be, the latter may further include a substantially tubular or even tapered section extension cooperating with the main member.

To be used as a therapeutic element in case of a infrarenal abdominal aorta aneurysm, the main member may consist of a “reversed Y”, the bifurcation of which is located in the lower portion thereof.

To be adapted to the pathology, the section of the expandable means of such an endoprosthesis may preferably be substantially tapered.

In order to help the practitioner in his/her treatment act, an endoprosthesis according to the invention may include radiopaque means. The latter may consist of a wire surrounding the upper portion of the expandable means or of a wire cooperating with the main member, on the whole length thereof.

In order to prevent any pinching of the main member upon applying the endoprosthesis, the lower portion of said main member may include a stent.

In order to return to an appropriate shape upon applying the endoprosthesis, the tubular main member may be surrounded by a plurality of wires composed of a shape memory material, the section of which is substantially smaller than that of the member.

The same may be true for the lower distal portion of the tubular main member of an extension which may advantageously include wires made of a wavy shape memory material which surrounds said lower portion of the member of said extension.

In order to limit the risk of embolism if the stent cooperating with the upper distal portion of the main member of an endoprosthesis according to the invention is positioned at an arterial or venous junction, said stent may advantageously include one or more openings facilitating a side blood flow.

### BRIEF DESCRIPTION OF THE DRAWINGS

Further features and advantages will appear more clearly from reading the following description and examining the supporting figures, among which:

FIG. 1 shows a partial view of the vascular system of a sound subject or of a patient suffering from an abdominal aorta aneurysm;

FIG. 2 shows an angioplasty process using a known endoprosthesis;

FIG. 3 describes an endoprosthesis according to the invention.

FIG. 4 describes an advantageous embodiment of a stent cooperating with the upper distal portion of the main member of the endoprosthesis according to the invention;

FIGS. 5 and 6 respectively show two advantageous embodiments integrating a stent such as the one described while referring to FIG. 4.

### DETAILED DESCRIPTION OF THE INVENTION

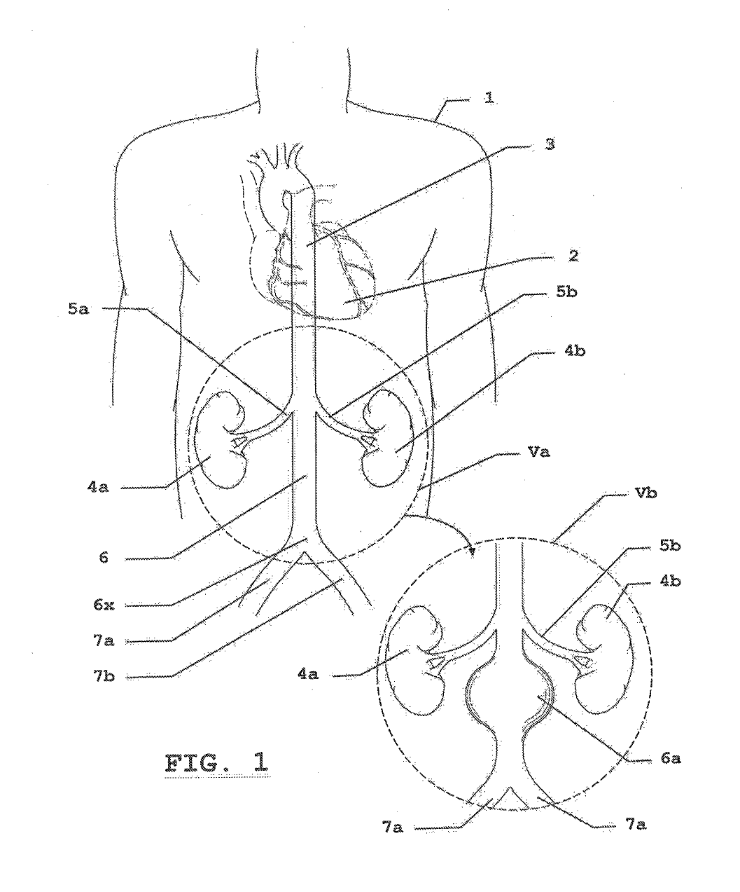

FIG. 1 shows a partial view of the vascular system of a sound subject 1 and that of a patient suffering from an abdominal aorta aneurysm (partial view Vb on the right of FIG. 1). A subject's aorta thus originates at the heart 2 and extends in the thoracic aorta 3 and then in the so-called abdominal aorta 6 downstream of the renal arteries 5a and 5b which respectively lead to the kidneys 4a and 4b. The infrarenal abdominal aorta of a sound subject has substantially parallel walls. The lower portion thereof further shows a bifurcation 6x to create the common iliac arteries 7a and 7b.

As shown in the partial view Vb on the right of FIG. 1, the infrarenal abdominal aorta 6 may suffer from a pathology known as an aneurysm. Said abdominal aorta deforms so as to create (downstream of the renal arteries and upstream of the common iliac arteries)—an aneurysmal sac, the development of which is inevitable and difficult to predict.

A known therapy consists of making an angioplasty. An endoprosthesis is then positioned within the pathologic aorta so that blood stops circulating in the aneurysmal sac but circulates through said endoprosthesis.

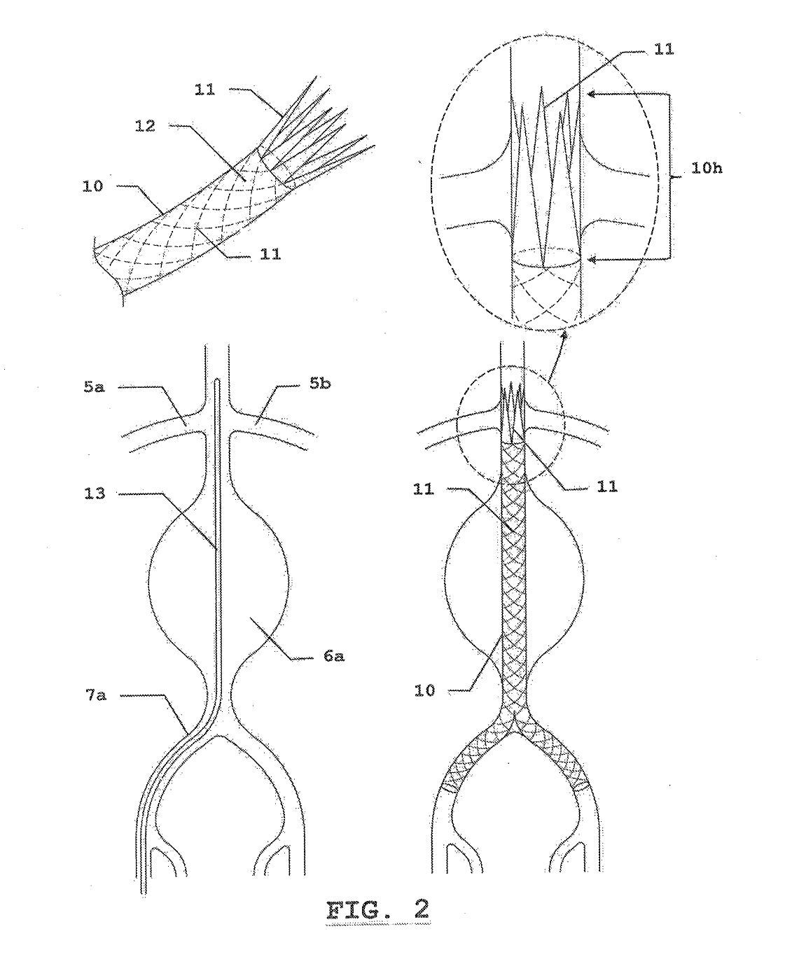

FIG. 2 shows an angioplasty process for an abdominal aorta. Thus as an example, a sheath 13 is introduced into a femoral artery in a patient's inguinal fold. Such sheath 13 moves forward inside a common iliac artery 7a and then through the aneurysmal sac 6a of the abdominal aorta 6 upstream of the renal arteries 5a and 5b. Such sheath 13 includes an endoprosthesis 10, which, as per the example disclosed while referring to FIG. 2, automatically unfolds upon removal of the sheath, under the spring effect of a stent 11 within the endoprosthesis covered with a membrane 12 and held in a compressed state by the sheath. Such a stent is made of a metal mesh generally made of chrome and cobalt alloy. According to a known technique, it is possible to unfold an endoprosthesis the shape of which is substantially that of a “reversed Y” so that the blood from the thoracic aorta can circulate through the endoprosthesis up to the renal arteries 5a, 5b, then to the common iliac arteries 7a, 7b. Blood circulates from the thoracic aorta to the renal arteries through the upper distal portion 10h of the endoprosthesis, a portion where the stent is not covered by the membrane 12 of the main member of the endoprosthesis. As a matter of fact, such membrane—generally woven from polyester—covers said stent on the whole length of the main member 12, except for said upper distal portion 10h of the endoprosthesis. Blood can thus freely circulate through the metal mesh of the stent 11 at the upper distal portion of the endoprosthesis 10h. On the other hand, downstream of said renal arteries, the membrane of the main member 12 prevents all blood circulation within the aneurysmal sac. The shape of the main member which is substantially bifurcated in a substantially “reversed Y shape” makes it possible to provide two lower distal portions adjusted respectively to the anatomy of the patient's common iliac arteries.

As an alternative solution, positioning an endoprosthesis including a polyester-based main member and including a stent essentially cooperating with said main member at the upper distal portion of said member is also known. As mentioned above, the main member of such an endoprosthesis may have the shape of a “reversed Y” in order to carry in fine blood from the thoracic aorta up to the common iliac arteries. This endoprosthesis is different from the preceding one in that the unfolding of the member and the stent is not automatic upon removing the sheath stressing the endoprosthesis. Such unfolding occurs using a balloon within the endoprosthesis. A controlled inflation of the latter makes it possible to push apart the stent mesh, so that it can be sufficiently deformed to adhere upstream of the renal arteries to the sound walls of the pathologic aorta. Such endoprosthesis shall be preferred more particularly in case of a loss of parallelism of the walls upstream of the aneurysmal sac or when the section of said aorta is not regular. Besides, using a balloon-expandable stent makes it possible to reduce the range of prostheses required to be able to meet the needs of the majority of patients. The balloon makes it possible to control the expansion of the stent and facilitates the application of an endoprosthesis in a large number of cases. On the contrary, an automatically expandable stent generally requires a larger range of endoprostheses so as to provide for various expansion and power ranges for the opening of the stent in order to provide, more particularly, the anchoring of the endoprosthesis in the wall of the pathologic artery.

Whatever the stent expansion mode, an endoprosthesis is positioned so that the stent upstream of the member 12 lets blood circulate from the thoracic aorta to the renal arteries. As for the main member, it prevents any rush of blood within the aneurysmal sac.

Both types of endoprostheses have proven their efficiency. However, the little predictable development of the pathology can cause a loss of tightness between the outer wall of the endoprosthesis and the inner wall of the abdominal aorta upstream of the aneurysm. Such a loss of tightness may result from an unwanted migration of the endoprosthesis or a deformation of the sound portion of the abdominal aorta upon the application of the endoprosthesis which now suffers from the same pathology. Endoleaks can thus occur, which cause serious complications for the patient. Then it becomes indispensable and urgent to remove the failing endoprosthesis and to apply a new one.

The invention addresses all the disadvantages of known endoprostheses.

As for known endoprostheses, the endoprosthesis according to the invention will be described while preferably referring to an abdominal aortic aneurysm. However, the invention applies to any endoprosthesis used in angioplasty. The main member thereof could thus be only and substantially tubular without any bifurcation(s).

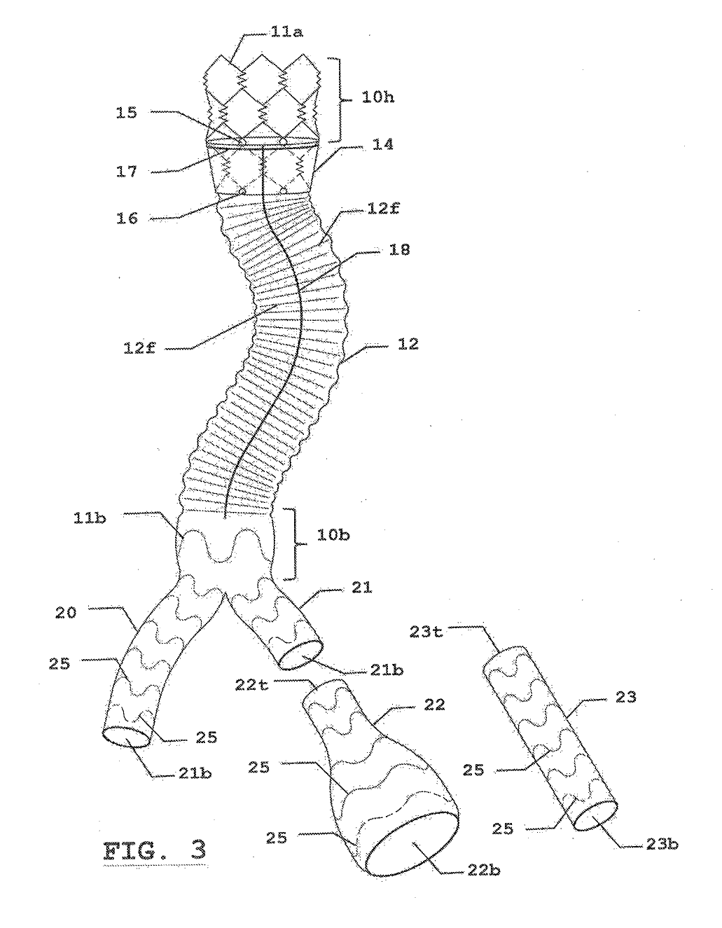

FIG. 3 discloses a preferred embodiment of an endoprosthesis according to the invention and more particularly adapted to the abdominal aortic aneurysm pathology.

Such a vascular endoprosthesis 10 according to the invention includes a substantially tubular main member 12, the upper distal portion of which cooperates with a stent 11a. According to a preferred embodiment, said stent is expandable, using an inner balloon (not shown). It shows a metal mesh (preferably made of a chrome-cobalt-nitinol-based alloy, and even stainless steel). The mesh is such that, upon inflating the balloon, the stent expands without retracting. As an example, such a stent may comply with the one described in the document U.S. Pat. No. 7,357,813, which is incorporated by reference.

The main member 12 is preferably made of a woven fabric, for instance polyester. The structure thereof is thus little or non expandable and provides a correct tightness. According to a particular embodiment, said member may be wavy because of the utilisation of one or more polyester wires 12f which surround said member 12. The section of said polyester wires 12f is substantially smaller than that of the member 12. Using such wires enables the member 12 to have a “shape memory” and thus to return to its tubular shape upon unfolding of the endoprosthesis. Any other shape memory material (for example a polymer tolerated by living beings) could be used, as an alternative solution. This technique facilitates the first passage of blood through said member just after the application of the endoprosthesis. Still in order to facilitate the first utilisation of the endoprosthesis, the invention provides that a secondary stent 11b could be provided, preferably in the lower portion 10b of the main member. According to the preferred embodiment disclosed while referring to FIG. 3, this stent is auto-expandable and preferably made of a wire (or a plurality of wires) made of a shape memory material, such as nitinol, for example, with such wire(s) cooperating with the lower portion of the main member. As an alternative solution, the stent 11b can be expanded by a balloon, like stent 11a. The height of stent 11b can be less than that of stent 11a.

As a matter of fact, the stent 11a is used not only for fixing the endoprosthesis to the wall of the aorta upstream of the renal arteries but also the height thereof must be sufficient to enable a free blood circulation through the stent mesh, in order to irrigate such renal arteries. Advantageously, the height of the stent may be between three and six centimetres. In the case of an endoprosthesis according to the invention, the fixing of which would be infrarenal (i.e. downstream of the renal arteries), the height of the stent 11a could be reduced.

In the exemplary application described while referring to FIG. 3, the endoprosthesis is intended for the angioplasty of the abdominal aorta. The main member 12 thus has a bifurcation and two lower distal portions. Such distal portions 20 and 21 are intended for cooperating with the patient's common iliac artery. Traditionally, the distal portion 20 corresponding to the common iliac artery through which the sheath including the endoprosthesis has been introduced may be longer than the second one. Such reversed “Y” shaped structure of the main member 12 depends on the angioplasty to be executed. The main member may be simply tubular in other exemplary applications.

According to the endoprosthesis described while referring to FIG. 3, the secondary stent 11b is preferably positioned at the bifurcation. According to the invention, other secondary stents could be provided for cooperating with the lower distal portions of the member 12.

In order to remedy the drawbacks of known endoprostheses, any endoprosthesis according to the invention includes expandable fabric means 14 covering the lower portion of the stent 11a and cooperating with the upper distal portion of the main member 12.

Such means may consist of an expandable ring made of a knitted material including a resilient synthetic fibre of the elastane type, for instance. Other fibres could be used. It is sufficient that the ring can be sufficiently deformed to follow the expansion of the stent 11a without for all that retracting in height.

The texture of such means 14 brings an increased tightness to the endoprosthesis anchoring point or more exactly to the point of contact of said means 14 and the inner wall of the abdominal aorta downstream of the renal arteries and upstream of the aneurysmal sac. Besides, the material naturally adheres to the wall of the artery such that an unwanted migration of the endoprosthesis is prevented. Advantageously, said means 14 is fixed 15—for example sewn—to the metal mesh of the stent 11a in order to ensure a correct behaviour. The means 14 is further fixed 16 in order to cooperate with the upper distal portion of the main member 12.

The dimensions, shapes and sections of the elements of an endoprosthesis according to the invention are defined so as to conform with the patient's anatomy and with situations of angioplasties. According to the example illustrated in FIG. 3, the main member 12 may have an average length—determined empirically or statistically—of the order of 8 cm. The height of the stent 11a can be approximately 3 cm to enable a correct anchoring and let blood circulate from the aorta to the renal arteries. The height of the seal ring 14 may preferably be 10 to 15 mm. The section of the main member 12—more particularly the upper distal portion cooperating with the means 14—is determined in order to be adapted to a majority of subjects. In order to ensure a correct resilience and expansion of the elements (stents 11a, 11b and ring 14), the invention advantageously provides that the section of the stents can increase between 22 and 28 mm for a first endoprosthesis and between 28 and 34 mm for a second one. In the first case, the section of the main member is substantially 22 mm and 24 mm for the second arrangement. Other alternative solutions could further be considered.

In order to be able to extend the distal portions of a main member (portions 20 and 21 according to the example of FIG. 3), the invention provides one or more substantially tubular extensions—made of a material similar to that of the main member—intended for cooperating with said distal portions of the main member. FIG. 3 thus discloses two types of extensions 22 and 23. The first type of extension 23 is substantially tubular, and the sections of the distal portions 23t, 23b are substantially identical. Such an extension may be several centimetres long so as to conform with the patient's anatomic constraints. Statistically, it seems possible to select a length of 5 cm. Such an extension may have a section of the same order as the section of the distal portion 21b of the main member to be extended. A section of the order of 10 mm can thus be advantageously chosen.

FIG. 3 discloses an alternative solution wherein the extension 22 has a tapered section. Thus, the invention provides a somewhat flared extension, i.e. 10 to 12 mm for a first distal portion 22t and 16 to 22 mm for the second one 22b. Whatever the selected extension, said extension should preferably be inserted into the distal portion of the member to be extended on a depth of the order of 30 mm. Other configurations and arrangements could however be devised according to the invention.

In addition, the latter provides that the lower distal portion(s) 20, 21 of the main member and/or the extensions 22 or 23 may include one or several wavy nitinol 25 wire(s) surrounding said lower portions of the member 12 and the extensions. Such nitinol wires are sewn on the inner or outer walls of the elements (main member and/or extensions). According to a preferred embodiment, the wires are sewn on the outer wall of the element. Such arrangement makes it possible to prevent the possible occurrence of a thrombus caused by a prolonged contact of blood, the flow of which may be low, with said wires 25. However,—as shown in FIG. 3—a last row of wires 25—the one surrounding a distal portion—sewn on the inner wall can be provided for reinforcing the technical effect generated by the shape memory wires.

Polymer- or any other material-based wires with a shape memory could be used in addition to or as a substitute for nitinol. Using the means 25 makes it possible to ensure that the lower portion of the endoprosthesis and possibly of the extensions will not be pinched or be an obstacle to the passage of blood upon the first blood flow. The straight or tapered section of the extensions makes it possible to conform with the patient's anatomy.

The expandable material of the means 14 for ensuring a correct tightness must not be an obstacle to the blood irrigation, more particularly of the renal arteries in the case of a supra-renal endoprosthesis as described while referring to FIG. 3. In order to enable the practitioner to correctly position the endoprosthesis and offer him/her a precious help to ensure that the means 14 will not be an obstacle to said renal arteries, the invention provides that the means 14 can include a (gold- or platinum-based, for instance) radiopaque wire 17 surrounding the upper portion of said means 14. The invention also provides that the main member 12 of an endoprosthesis according to the invention may include a radiopaque wire 18 cooperating with the main member on the whole length thereof. Other radiopaque means, although structurally different from wires, but liable to fulfil the same functions as the wires 17 and 18 could be used. Using radiopaque means (17 or 18) makes it possible to detect any incorrect position of the means 14, but also any inconvenient pinching or torsion of the main member. Such means further could cooperate with the extensions as described above. Thus, a practitioner using radiological means during the angioplasty, can take advantage of a precious help for checking the relevance of the treatment act.

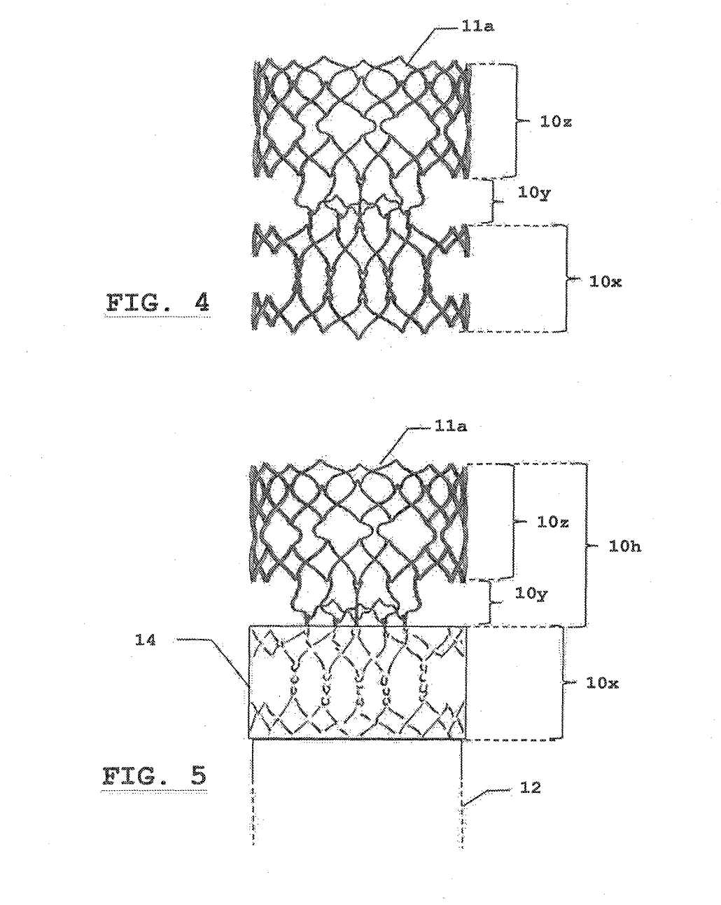

FIGS. 4, 5 and 6 disclose a stent preferably adapted to cooperate with the upper distal portion of a main member of an endoprosthesis according to the invention.

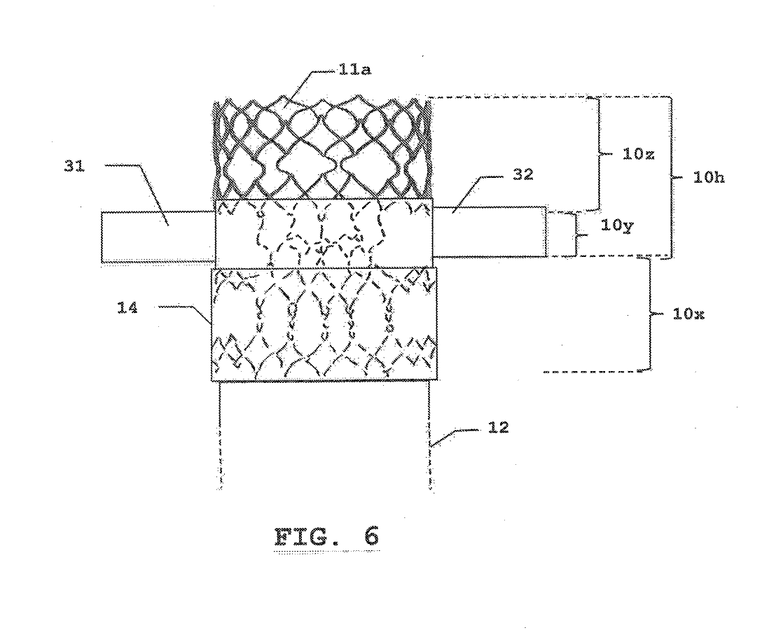

The stent 11a illustrated in FIG. 4 shows a particularly advantageous configuration more particularly when said stent must unfold at a junction between the main vessel (submitted to an angioplasty) and one or several secondary vessels. For this purpose, the stent 11a includes three contiguous cylindrical portions, respectively: 10x for the proximal (or lower) portion, 10y for the central portion and 10z for the distal (or upper) portion. As will be mentioned hereunder while referring to FIG. 5, such a stent may advantageously be used as a stent for anchoring a supra-renal endoprosthesis treating an abdominal aorta aneurysm such as the endoprosthesis described above while referring to FIG. 3. The stent of such an endoprosthesis may unfold (automatically or using a balloon) at the junction of the renal arteries with the abdominal aorta. The central portion 10y of the stent 11a is so arranged as to have one or several opening(s) to facilitate a side flow of blood (for example to the renal arteries). The stent mesh is thus “lightened” in the central portion 10y thereof with respect to the proximal 10x and distal 10z portions. However, said mesh 10y remains advantageously resistant—in spite of said openings—to ensure a reliable connection between the distal 10z and proximal 10x portions. The distal portion 10z of the stent may advantageously be so arranged as to develop a radial force higher than the one developed by the other two portions 10x and 10y in order to maximize a “formwork” function or optimize the anchoring of the prosthesis upon the (automatic or balloon-assisted) unfolding of the stent.

FIG. 5 discloses a partial enlargement of a supra-renal endoprosthesis similar to the one described above while referring to FIG. 3 and the stent 11a of which is complying with the one illustrated while referring to FIG. 4. According to this example, the height of the stent 11a is advantageously chosen to be substantially equal to 4.5 centimetres. Generally said height may vary between three and six centimetres for the stent of such an endoprosthesis. The junction 10h of the distal 10z and central 10y portions of the stent 11a is covered neither by the main member 12, nor by the tightness means 14. The lower cylindrical portion 10x is covered by the means 14 providing a correct tightness against the wall of the aorta located under the renal arteries. The lower portion 10x of said stent 11a thus shows through in dotted lines in FIG. 5. The arrangement of the central portion 10y of the stent 11a facilitates the circulation of blood at the junction of the renal arteries and the abdominal aorta. This advantageous arrangement prevents any obstruction of the blood flow circulating in the renal arteries. The risk of micro-clots being transported to the kidneys is thus reduced. The upper (or distal) portion 10z of the stent may advantageously be so arranged as to develop a radial force higher than the one developed by the other two portions 10x and 10y and optimize the anchoring of the prosthesis upon the (automatic or balloon-assisted) unfolding of the stent.

FIG. 6 describes a partial view of the distal or upper portion of a second supra-renal endoprosthesis according to the invention and integrating a stent such as described above in FIG. 4. In order to further facilitate the circulation of blood in the renal arteries, the main member 12 of the endoprosthesis so extends (with regard to the endoprosthesis described while referring to FIG. 3) as to cover the central portion 10y of the stent 11a. According to a first embodiment, the upper distal portion of said member 12 cooperating with the central portion 10y of the stent 11a has side openings respectively cooperating with two extensions 31 and 32 provided for entering said renal arteries respectively. The materials used for making said extensions 31 and 32 may advantageously be similar to those used for making the main member 12 of the endoprosthesis. As an alternative solution, the main member 12 of the endoprosthesis is made of a single piece including the tubular member and both extensions 31 and 32. Other configurations according to the invention could be considered (only one extension of the main member or more than two extensions) depending on the application desired, the patient's morphology or the configuration of the pathologic vessel. The central portion 10y of the stent positioned at an arterial or venous junction just has to have a mesh with openings aiming at facilitating blood flow.

The invention has been disclosed while referring preferably with an abdominal aorta aneurysm in a human being. The invention naturally applies to any other endoprosthesis having a main member and a stent for which it is essential to prevent any risk of endoleak between the outer wall of the endoprosthesis and the inner wall of the pathologic artery.

1. A vascular endoprosthesis including a substantially tubular main member an upper distal portion of which cooperates with a stent, wherein said endoprosthesis includes expandable fabric means covering a lower portion of said stent and cooperating with the upper distal portion of said member.

2. The endoprosthesis according to claim 1, wherein the tubular member comprises woven polyester.

3. The endoprosthesis according to claim 1, wherein the stent is expandable, non-retractable and consists in a chrome-cobalt- and nitinol-based metal mesh.

4. The endoprosthesis according to claim 1, wherein the stent is expandable, non-retractable, and comprises a stainless steel metal mesh.

5. The endoprosthesis according to claim 1, wherein the expandable means comprises a knitted material including a resilient synthetic fibre.

6. The endoprosthesis according to claim 1, further including a substantially tubular extension cooperating with the main member.

7. The endoprosthesis according to claim 6, wherein the extension has a tapered section.

8. The endoprosthesis according to claim 1, wherein the main member comprises a “reversed Y” shape, the bifurcation of which is located in the lower portion thereof.

9. The endoprosthesis according to claim 1, wherein the section of the expandable means is substantially tapered.

10. The endoprosthesis according to claim 1, including radiopaque means.

11. The endoprosthesis according to claim 10, wherein the radiopaque means comprises a wire surrounding the upper portion of the expandable means.

12. The endoprosthesis according to claim 10, wherein the radiopaque means comprises a wire cooperating with the main member on the whole length thereof.

13. The endoprosthesis according to claim 1, wherein the lower portion of the main member includes a stent.

14. The endoprosthesis according to claim 1, wherein the tubular main member is surrounded by a plurality of wires composed of a shape memory material, a section of which is substantially smaller than that of the member.

15. The endoprosthesis according to claim 1, wherein the lower distal portion of the tubular main member includes wires composed of a wavy shape memory material surrounding said lower portion of the member.

16. The endoprosthesis according to claim 6, wherein the extension of the tubular main member includes wires composed of a wavy shape memory material surrounding said extension.

17. The endoprosthesis according to claim 1, wherein the stent cooperating with the upper distal portion of the main member, includes one or several openings facilitating a side blood flow.
